(19) United States
(12) Patent Application Publication
(10) Pub. No.: US 2023/0383228 A1
(43) Pub. Date: Nov. 30, 2023

(54) Barrier Apparatus For Cell Culture And Methods Of Cell Culture Using The Barrier Apparatus

(71) Applicant: THE REGENTS OF THE UNIVERSITY OF MICHIGAN, Ann Arbor, MI (US)

(72) Inventors: Stephen E. FEINBERG, Ann Arbor, MI (US); James Brian WASHINGTON, Saline, MI (US); Roger W. MULDER, Dexter, MI (US); Allen SAMUELS, Ann Arbor, MI (US); Cynthia L. MARCELO, Ann Arbor, MI (US)

(73) Assignee: THE REGENTS OF THE UNIVERSITY OF MICHIGAN, Ann Arbor, MI (US)

(21) Appl. No.: 18/202,602

(22) Filed: May 26, 2023

Related U.S. Application Data
(60) Provisional application No. 63/346,345, filed on May 27, 2022.

(51) Int. Cl. C12M 1/42; C12M 1/00; C12N 5/071

(52) U.S. Cl. CPC C12M 35/04; C12M 23/38; C12M 29/20; C12N 5/0629

(57) ABSTRACT

An apparatus (e.g., for cell culture for in vitro growth of tissues and/or organs) includes a base, a plate on the base, and a removable barrier component on the plate. The removable barrier component is configured to at least partially define a first region. The removable barrier component includes a bottom surface and a top surface. The bottom surface configured to be in contact with the plate. The top surface is opposite the bottom surface. The press is configured to engage the top surface of the removable barrier component to apply a predetermined pressure to the removable barrier component. The removable barrier component is between the press and the plate. A joint between the plate and the removable barrier component may be substantially impermeable to live cells. The apparatus may further include a removable cover that engages the base to enclose the plate, the removable barrier component, and the press.

### CROSS-REFERENCE TO RELATED APPLICATIONS

This application claims the benefit of U.S. Provisional Application No. 63/346,345, filed on May 27, 2022. The entire disclosure of the above application is incorporated herein by reference in its entirety.

### GOVERNMENT SUPPORT

This invention was made with government support under DE026915 awarded by the National Institutes of Health. The government has certain rights in the invention.

### FIELD

The present disclosure relates to a barrier apparatus for cell culture and methods of cell culture using the barrier apparatus.

### BACKGROUND

This section provides background information related to the present disclosure which is not necessarily prior art.

Cell culture is used in in a variety of fields for research and/or medical purposes. For example, cell culture may be used in developmental biology, regenerative biology (e.g., wound healing, tissue engineering, regenerative therapy), cellular migration (e.g., normal and metastasis), cell-cell crosstalk (e.g., tissue regulation in the context of disease and/or normal conditions), and drug development, among others. Cell culture is a major technique utilized in the academic research environment. These in vitro assays have allowed for the studying of cell types outside of the complex biological environment where they originate. Cell culture may also be used for in vitro growth of tissues and/or organs to be transferred in vivo.

### SUMMARY

This section provides a general summary of the disclosure, and is not a comprehensive disclosure of its full scope or all of its features.

In various aspects, the present disclosure provides an apparatus including a base, a plate, and a removable barrier. The plate is on the base. The removable barrier component is on the plate. The removable barrier component is configured to at least partially define a first region. The removable barrier component includes a bottom surface and a top surface. The bottom surface configured to be in contact with the plate. The top surface is opposite the bottom surface. The press is configured to engage the top surface of the removable barrier component to apply a predetermined pressure to the removable barrier component. The removable barrier component is between the press and the plate.

In one aspect, the removable barrier component is configured to at least partially define a plurality of regions including the first region.

In one aspect, the removable barrier component further includes a transverse wall between the first region and a second region of the plurality of regions.

In one aspect, the transverse wall defines a thickness of greater than or equal to about 0.1 mm to less than or equal to about 50 mm.

In one aspect, the thickness is greater than or equal to about 0.5 mm to less than or equal to about 5 mm.

In one aspect, the transverse wall defines a port fluidly connecting the first region and the second region.

In one aspect, the removable barrier component further includes a peripheral wall. The peripheral wall surrounds the first region and the second region.

In one aspect, the first region and the second region are different in terms of projected area, volume, shape, or a combination thereof.

In one aspect, the second region is nested within the first region.

In one aspect, the removable barrier component at least partially further defines a third region.

In one aspect, the apparatus further comprises a plurality of removable barrier components. The removable barrier components of the plurality of removable barrier components are configured to be independently and sequentially disposed on the plate, The plurality of removable barrier components includes the removable barrier component and a second removable barrier component. The second removable barrier component at least partially defines a second region different from the first region.

In one aspect, the second region at least partially surrounds the first region in first, second, and third orthogonal directions.

In one aspect, the removable barrier component defines a plurality of wedge-shaped regions including the first region.

In one aspect, the removable barrier component defines a plurality of apertures. The plurality of apertures includes a first aperture defining the first region.

In one aspect, the apparatus further includes a removable cover defining an interior region. The removable cover is configured to engage the base to enclose the plate, the removable barrier component, and the press within the interior region.

In one aspect, the removable cover defines a vent configured to permit air transfer to and from the interior region of the removable cover.

In one aspect, the base defines a vent configured to permit air transfer to and from the interior region of the removable cover.

In one aspect, the apparatus further includes a barrier cap. The barrier cap is configured to be between the removable barrier component and the press. The barrier cap is configured to align the press, the removable barrier component, and the plate along a press axis.

In one aspect, the barrier cap is configured to engage an entirety of the top surface of the removable barrier component.

In one aspect, the barrier cap defines an opening in fluid communication with the first region.

In one aspect, the removable barrier component and the barrier cap are an integrally formed unitary structure.

In one aspect, the barrier cap defines a recess configured to receive at least a portion of the removable barrier component.

In one aspect, the base defines a recess. The plate is at least partially in the recess.

In one aspect, the plate includes glass, plastic, or a combination of glass and plastic.

In one aspect, a joint between the plate and the removable barrier component is configured to be substantially impermeable to live cells.

In one aspect, the plate and the removable barrier component are configured to receive a pliable material therebetween.

In one aspect, a joint between the pliable material and the removable barrier component is configured to be substantially impermeable to live cells.

In various aspects, the present disclosure provides an apparatus for creating cell cultures. The apparatus includes a base, a press, and a removable cover. The base is configured to support a plate and a removable barrier component that cooperate to define a region for receiving cells. The plate is between the base and the removable barrier component. The press is configured to apply pressure to the removable barrier component such that a joint between the plate and the removable barrier component is substantially impermeable to live cells. The removable barrier component is between the press and the plate. The removable cover defines an interior region. The removable cover is configured to engage the base to enclose the press, the plate, and the removable barrier component within the interior region.

In various aspects, the present disclosure provides a method of creating a cell construct. The method includes applying a first predetermined pressure to a first barrier component using a press. The first barrier component is between the press and a plate. The plate is between the first barrier component and a base. The press is coupled to the base. The method further includes, concurrently with the applying, creating a culture by seeding a first population of cells in a first region at least partially defined by the plate and the first barrier component. The method further includes concurrently with the applying, retaining the first population of cells in the first region for a first predetermined period of time. The method further includes removing the first barrier component from the plate. The method further includes forming the cell construct by maturing the culture for a second predetermined period of time.

In one aspect, the creating further includes seeding a second population of cells in a second region of the plate. The second region is at least partially defined by the plate and the first barrier component. The second region is separated from the first region by a wall of the first barrier component.

In one aspect, the method further comprises, after the removing, placing a second barrier component in contact with the plate. The second barrier component defines a second region at least partially surrounding the first region. The method further includes applying a second predetermined pressure to the second barrier component using the press. The second barrier component is between the press and the plate. The method further includes, concurrently with the applying the second predetermined pressure. The method further includes creating a coculture by seeding a second population of cells in the second region. The method further includes, concurrently with the applying the second predetermined pressure, retaining the second population of cells in the second region for a third predetermined period of time. The method further includes removing the second barrier component from the plate.

In one aspect, the culture includes a tissue matrix, a multi-layered tissue structure, or an organ.

In various aspects, the present disclosure provides a method of creating a mucocutaneous construct (MCC). The method includes applying a predetermined pressure to a barrier component using a press. The barrier component is between the press and a plate. The plate is between the barrier component and a base. The press is coupled to the base. The method further includes, concurrently with the applying, creating a coculture. Creating a coculture includes seeding a first population of skin keratinocytes in a first region in a first liquid phase. The first region is at least partially defined by the plate and the barrier component. Creating the coculture further includes seeding a second population of mucosa keratinocytes in a second region in the first liquid phase. The second region is at least partially defined by the plate and the barrier component. The second region is separated from the first region by a wall of the barrier component. The method further includes concurrently with the applying, retaining the coculture in the first liquid phase for a predetermined period of time. The method further includes removing the barrier component from the plate. The method further includes after the removing, retaining the coculture in a second liquid phase for a second predetermined period of time. The method further includes forming the MCC by maturing and stratifying the coculture by retaining the coculture in an air-liquid phase for a third predetermined period of time. The MCC includes skin cells and mucosa cells.

In one aspect, the method further includes prior to the applying, disposing a non-immunogenic acellular dermal matrix on the plate in the first liquid phase such that the non-immunogenic acellular dermal matrix is between the plate and the barrier component.

In one aspect, a joint between the plate and the wall of the barrier component is configured to be substantially impermeable to live cells during the applying.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1C relate to a barrier apparatus according to at least one example embodiment; FIG. 1A is a perspective view of the barrier apparatus; FIG. 1B is a perspective view of the barrier apparatus having a cover removed; and FIG. 1C is a perspective view of a base of the barrier apparatus.

FIG. 2 is a perspective view of a housing for a barrier apparatus according to at least one example embodiment.

FIGS. 3A-3C depict a barrier component according to at least one example embodiment. FIG. 3A is a top perspective view; FIG. 3B is a bottom perspective view; and FIG. 3C is a cross sectional view along line 3C-3C of FIG. 3A.

FIG. 4 is a top perspective view of a barrier component according to at least one example embodiment, the barrier component defining ports.

FIG. 5 is a perspective view of a barrier component according to at least one example embodiment.

FIGS. 6A-6B relate to a barrier component according to at least one example embodiment, the barrier component defining apertures; FIG. 6A is a top perspective view of the barrier component; and FIG. 6B is a bottom perspective view of the barrier component.

FIG. 7 is a top elevation view of a barrier component according to at least one example embodiment, the barrier component including nested regions.

FIGS. 8A-8D are top elevation views of a plurality of barrier components for creating a multi-laminate according to at least one example embodiment; FIG. 8A depicts a first barrier component; FIG. 8B depicts a second barrier component; FIG. 8C is depicts a third barrier component; and FIG. 8D depicts a fourth barrier component.

FIG. 9 is a perspective view of a barrier apparatus according to at least one example embodiment, the barrier apparatus depicted without a cover, the barrier apparatus including a barrier cap.

FIG. 10 is a perspective view of a barrier apparatus according to at least one example embodiment, the barrier apparatus depicted without a cover, the barrier apparatus including a barrier cap.

FIGS. 11A-11B depict a barrier cap according to at least one example embodiment; FIG. 11A is a top perspective view of the barrier cap; and FIG. 11B is a bottom perspective view of the barrier cap.

FIGS. 12A-12C relate to an integral barrier component-cap according to at least one example embodiment; FIG. 12A is a top perspective view of the barrier component-cap; FIG. 12B is a side elevation view of the barrier component-cap; and FIG. 12C is a bottom perspective view of the barrier component-cap.

FIG. 13 is a flowchart depicting a method of cell culture using a barrier apparatus according to at least one example embodiment.

FIGS. 14A-14F are schematic views depicting an implementation of the method of FIG. 13 according to at least one example embodiment. FIG. 14A illustrates a custom barrier component. FIG. 14B illustrates an alignment configuration including the custom barrier component of FIG. 14A, a barrier apparatus, a plate, and a pliable matrix.

FIG. 14C illustrates a live-cell-impermeable configuration created by applying a pressure to the alignment configuration of FIG. 14B. FIG. 14D illustrates a creation of a cell culture in the live-cell impermeable configuration of FIG. 14C. FIG. 14E illustrates a cell culture created in FIG. 14D. FIG. 14F illustrates a cell construct formed from the cell culture of FIG. 14E.

FIG. 15 is perspective view of a multi-laminate formed using the barrier apparatus and methods of at least one example embodiment.

FIG. 16 is perspective view of a multi-laminate formed using the barrier apparatus and methods of at least one example embodiment.

FIG. 17 is a photograph from an inverted phase microscope of a cell culture formed using the barrier apparatus and methods of at least one example embodiment according to at least one example embodiment.

Corresponding reference numerals indicate corresponding parts throughout the several views of the drawings.

### 1DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As described above, cell culture has numerous applications in both research and medical settings. Cell culture involves growing cells in controlled conditions. Preparation of cell cocultures involves growing distinct cell types in the same or similar conditions. One method of indirect coculture involves separating two regions by a porous membrane that reduces or inhibits movement of live cells between the two regions while permitting liquid to flow therebetween. The regions may be on different planes. Another method of indirect coculture involves the use of specialized connectable vessels. Different cell types may initially be grown in separate vessels and subsequently connected by a specialized adaptor to permit movement between the vessels.

Many cell culture methods are limited in terms of size, shape, and arrangement of different regions. For example, based on available coculture equipment, each region may be substantially the same size and shape as other regions. In some cases, interaction points between different regions may be limited, such as when a specialized adaptor is used to connect two regions. Coculture methods may also be limited in terms of region volume, which may be particularly challenging when larger volumes of analyte are needed, such as in identification of rare analytes in culture medium. Moreover, a distance between regions of a coculture is often preset based on the cultureware used and it cannot be changed. Accordingly, it may be difficult to create cocultures that mimic natural arrangements.

### Cell Culture Barrier Apparatus

At least one example embodiment relates to a barrier apparatus for cell culture. The barrier apparatus generally includes a base, a press or clamp fixed to the base, and a cover that cooperates with the base to enclose the press within an interior region. The interior region receives a plate or dish and a barrier component. The plate and barrier component cooperate to define one or more regions for cell growth. A pliable matrix may optionally be placed between the plate and the barrier component such that the pliable matrix would be in direct contact cell culture. The press applies a predetermined pressure to clamp the barrier component against the plate, thereby creating a joint or boundary between the plate and barrier component that is substantially impermeable to live cells. That is, the regions may be isolated from one another while the press engages the barrier component against the plate. In at least one example embodiment, the apparatus further includes a barrier cap that facilitates axial alignment of the press and the barrier component and/or a substantially uniform application of pressure to the barrier component.

The barrier apparatus may be customizable in terms of accommodating a variety of standard or custom plates or dishes and custom barrier components. Moreover, the barrier apparatus could be scaled up to accommodate growth of larger cell constructs. In at least one example embodiment, the plate may include or be part of standard lab equipment, such as a microscope slide or culture dish. In at least one example embodiment, the barrier component may be custom manufactured (e.g., three dimensional (3D) printed) to facilitate growth of a desired culture. The barrier component may include as many regions as desired, with each of the regions having a desired shape, placement, volume, footprint, and points of interaction. The regions may be the same as or different than other regions. In at least one example embodiment, a user may select a desired pressure to ensure that the live-cell-impermeable boundary is formed. The plate and the barrier component may be configured to be sanitized and reused, such as for certain research studies. In other example embodiments, the plate and barrier component are configured to be disposable, such as for certain medical applications.

In at least one example embodiment, the regions are coplanar. In at least one example embodiment, the barrier apparatus may facilitate growth of multiple cell types on a single plate or in a single culture dish without the cell types physically touching. The barrier component is removable from the plate. Accordingly, it is possible to remove the barrier component after a predetermined period of time to continue cell growth and/or maturation in different conditions (e.g., while permitting communication between the regions) on the same plate or dish. The barrier apparatus may be configured for single layer/two-dimensional (2D) culture or a multi-layer/3D culture.

The regions may be defined by one or more walls of the barrier. In at least one example embodiment, the adjacent regions are separated by a single wall. Accordingly, a thickness of the wall may define distances between different regions, and optionally different cell types. The barrier component can be manufactured (e.g., 3D printed) to have a wall thickness that facilitates regions being separated by a smaller distance than is possible with other cultureware. For example, distances small enough to mimic physiological conditions.

The cell construct that is created using the barrier apparatus may be used as a building block with other cell constructs and/or folded, twisted, rolled on to itself, or otherwise manipulated to connect two or more surfaces to create a more complex structure. Cell constructs may be used to form tissue matrices, multi-layered tissue structures, or organs, by way of example.

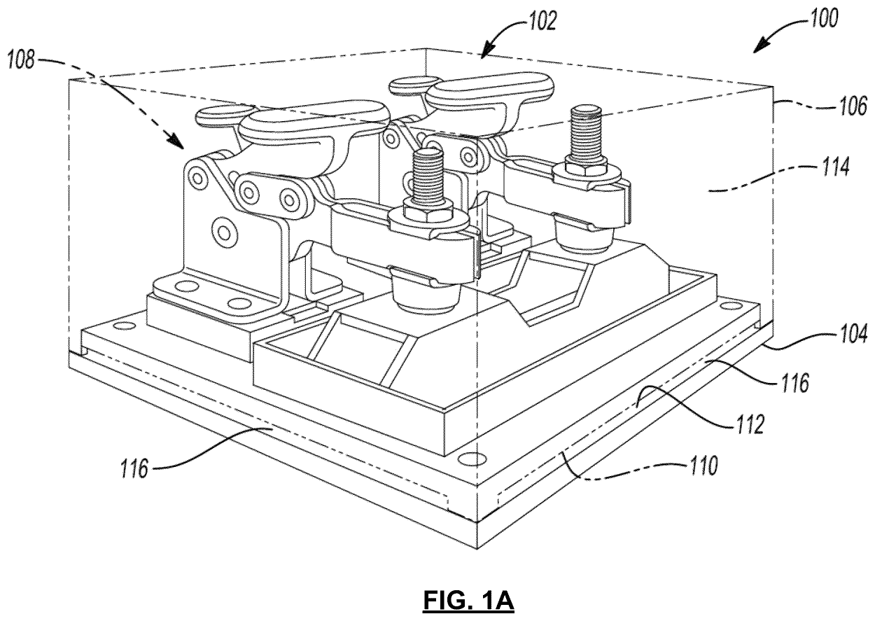

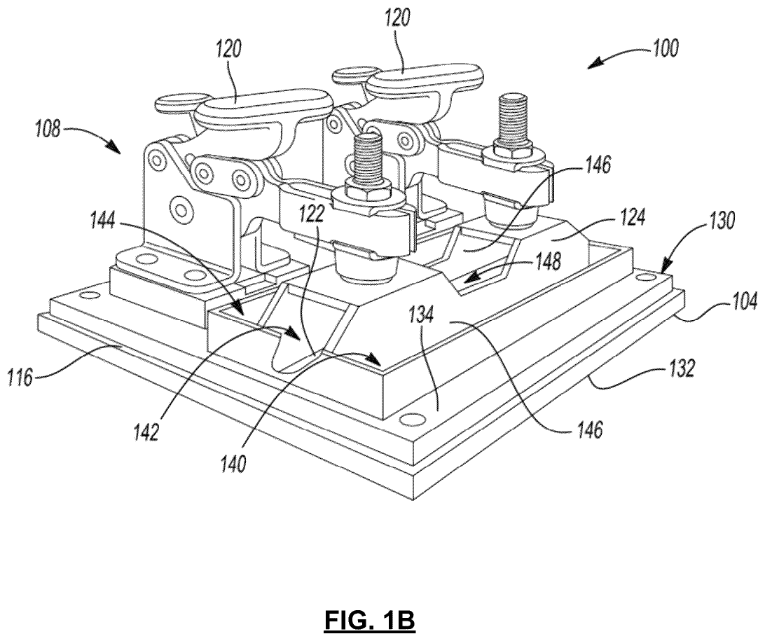

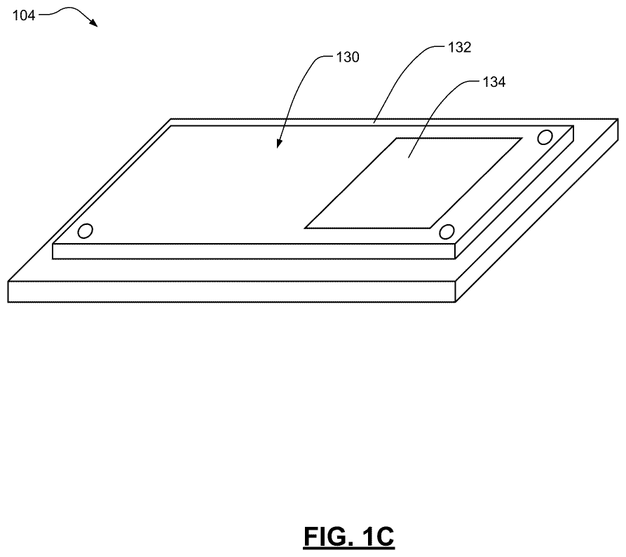

Referring to FIG. 1A, a barrier apparatus 100 according to at least one example embodiment is provided. The barrier apparatus 100 includes a housing 102. The housing 102 may include a base 104 and a removable cover or globe 106. The removable cover 106 cooperates with the base 104 to at least partially define an interior region 108.

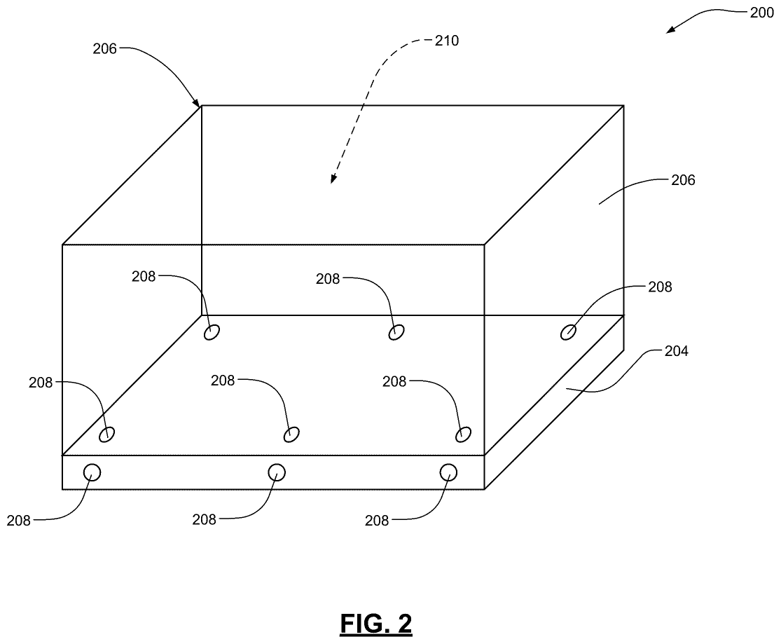

A bottom surface 110 of the cover 106 contacts (e.g., directly contacts) a top surface 112 of the base 104. In at least one example embodiment, a wall 114 of the cover 106 defines one or more openings or vents 116. The openings 116 are configured to permit fluid or gas transfer (e.g., passive airflow) to and from the interior region 108. In the example embodiment shown, the cover 106 defines an elongated rectangular opening on each of four sides of the housing 102. Additionally or alternatively to the openings 116 defined in the cover 106, a base of a housing may define openings or vents (see, e.g., vents 208 in FIG. 2).

With reference to FIG. 1B, the barrier apparatus 100 further includes one or more presses or clamps 120, a plate 122, and a barrier component 124. The cover 106 (shown in FIG. 1A) is configured to enclose the presses 120, the plate 122, and the barrier component 124 within the interior region 108. The plate 122 and the barrier component 124 may be removable, customizable, and/or disposable, as will be discussed in greater detail below. The plate 122 is configured to be disposed on the base 104. The barrier component 124 is configured to be disposed on the plate 122. The presses 120 are configured to apply pressure to the barrier component 124 such that a joint between the barrier component 124 and the plate 122 is substantially impermeable to live cells, as will be discussed in greater detail below.

### Housing

Returning to FIG. 1A, as discussed above, the housing 102 includes the base 104 and the cover 106, which cooperate to define the interior region 108. In at least one example embodiment, the cover 106 may be at least partially transparent. In at least one example embodiment, the cover 106 may be formed from or include poly(methyl methacrylate) (also referred to as acrylic).

Referring to FIG. 1C, in at least one example embodiment, the base 104 includes a main portion 130 and a flange 132. The flange 132 may be configured to contact the cover 106 (shown in FIG. 1A). In at least one other example embodiment, a base may be free of a flange. In at least one example embodiment, the base 104 is formed from or includes acrylonitrile butadiene styrene (ABS).

In at least one example embodiment, the top surface 112 of the base 104 defines a recess 134. The recess 134 may be configured to receive at least a portion of the plate 122 (shown in FIG. 1B) to align and/or stabilize the plate 122 with respect the press 120. In at least one other example embodiment, a portion of a base is free of a recess such that the plate sits on a continuous planar surface and a position of the plate is adjustable in horizontal and vertical directions.

At least one of the base 104 and the cover 106 may define one or more vents to permit fluid or gas (e.g., air) transfer to and from the interior region 108, as described above. In the example embodiment shown, the cover 106 defines the openings 116. With reference to FIG. 2, in at least one other example embodiment, a barrier apparatus 200 has a housing 202 including a base 204 and a cover 206. The base 204 defines a plurality of vents 208. The vents 208 are configured to permit fluid or gas transfer to and from an interior region 210 of the housing 202. The vents 208 may be apertures, as shown. Additionally or alternatively, vents 208 may be formed as channels in one or more surfaces of a base.

### Press

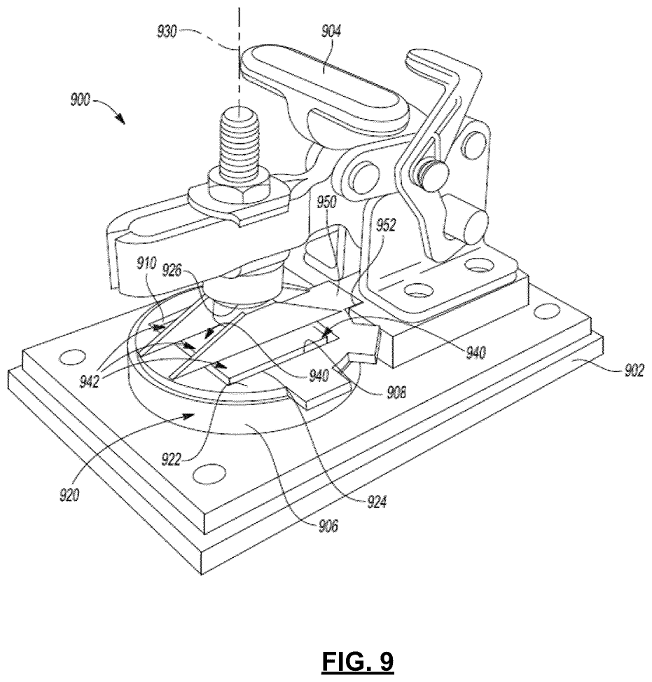

Returning to FIG. 1B, the presses 120 are coupled to the base 120. In at least one example embodiment, the presses 120 are coupled to the base 104 by a plurality of stainless steel bolts and washers (not shown). In the example embodiment shown, the barrier apparatus 100 includes two presses 120. In at least one other example embodiment, a barrier apparatus includes a single press (see, e.g., barrier apparatuses 900, 1000 of FIGS. 9, 10, respectively). In at least one other example embodiment, a barrier apparatus includes more than two presses.

The presses 120 may include mechanical, pneumatic, and/or hydraulic presses or clamps. In at least one example embodiment, each of the presses 120 includes a toggle clamp, such as a hold-down toggle clamp. The presses 120 may be formed from or include stainless steel. In at least one example embodiment, the barrier apparatus operatively connected to a control system for dynamic modification of pressure.

The presses 120 may be configured to apply a predetermined (or alternatively, desired) pressure to the barrier component 124. The pressure may be adjustable. The presses 120 may be configured to engage the barrier component 124 for a predetermined (or alternatively, desired) duration. In at least one example embodiment, the presses 120 are configured to apply substantially the same pressure throughout the entire predetermined duration. In at least one other example embodiment, the presses 120 are configured to apply variable pressure throughout the predetermined duration, such as when the presses 120 include pneumatic presses.

### Plate

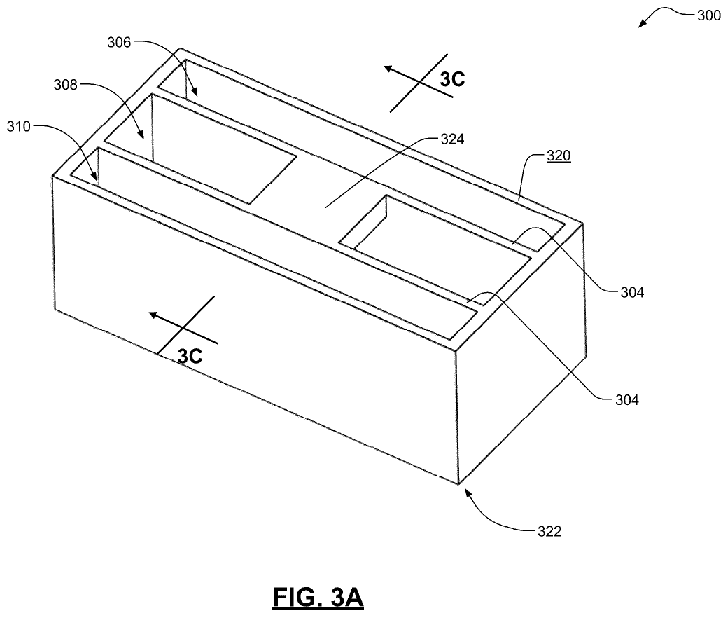

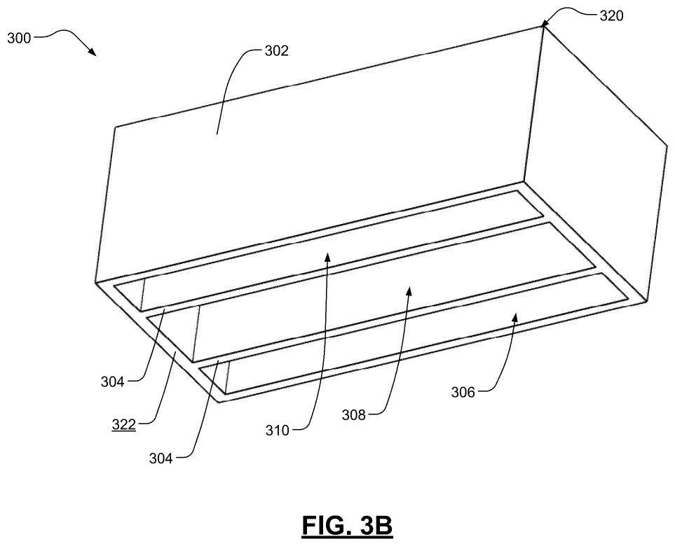

In at least one example embodiment, the plate 122 is configured to directly contact the base 104. The plate 122 may be disposed at least partially within the recess 134. The plate 122 may be removable from the base 104. The plate 122 is configured to be in contact (directly or indirectly) with the barrier component 124 to at least partially define a plurality of regions, as will be described below in relation to FIGS. 3A-7. In at least one example embodiment, the plate 122 is configured to be in direct contact with the barrier component 124.

In at least one example embodiment, the plate 122 may be configured to contain and/or support a pliable material such that the pliable material is disposed between the plate 122 and the press 120 (shown in FIG. 1B). In at least one example embodiment, the pliable material includes an acellular dermal matrix, a tissue matrix, or a combination thereof.

In at least one example embodiment, a size, shape, and material of the plate 122 are not particularly limited. For example, when a base is free of a recess (see, e.g., recess 134 of FIG. 1C), it may receive a range of sizes and shapes of plates. Accordingly, in at least one example embodiment, a user may employ any desired plate or vessel without need for specialized equipment. Moreover, in at least one example embodiment, the apparatus 100 is configured for use with standard or customized plates. The plate 122 may include glass, plastic, or a combination thereof. In at least one example embodiment, the plate is or is part of a microscope slide or a culture dish. The plate 122 may be configured to be sanitized and reused, or alternatively, disposable.

### Barrier Component

In at least one example embodiment, the barrier component 124 is removable, customizable, and/or disposable. As described above, the barrier component 124 is configured to cooperate with the plate 122 to one or more regions. In the example embodiment shown, the plate 122 and the barrier component 124 cooperate to define a first region 140, a second region 142, and a third region 144. The regions 140, 142, 144 may be at least partially separated from one another by transverse or inner walls 146 of the barrier component 124.

In at least one example embodiment, a joint 148 between the plate 122 and the barrier component 124 (e.g., the transverse walls 146) is substantially impermeable to live cells. That is, live cells generally cannot migrate between any of the regions 140, 142, 144.

In at least one example embodiment, the barrier component 124 and/resulting regions are customizable. For example, a user may define component size, component material number of regions, region shape, region footprint, region volume, region position, and/or points of interaction between different regions. The customizability of the barrier component 124 is facilitated by the ability of the housing 102 and the press 120 to uniformly apply the predetermined pressure to the barrier component 124 to create the impermeable joint 148. In at least one example embodiment, the apparatus 100 is free of restriction to particular cultureware.

The barrier component 124 may be configured to define one or more regions (e.g., greater than or equal to 2 regions, greater than or equal to 3 regions, greater than or equal to 4 regions, greater than or equal to 5 regions, greater than or equal to 10 regions, greater than or equal to 25 regions, or greater than or equal to 50 regions). Regions may have the same size or different sizes in terms of footprint and/or volume. In at least one example embodiment, volume of a region is controlled modifying a height of the barrier component 124.

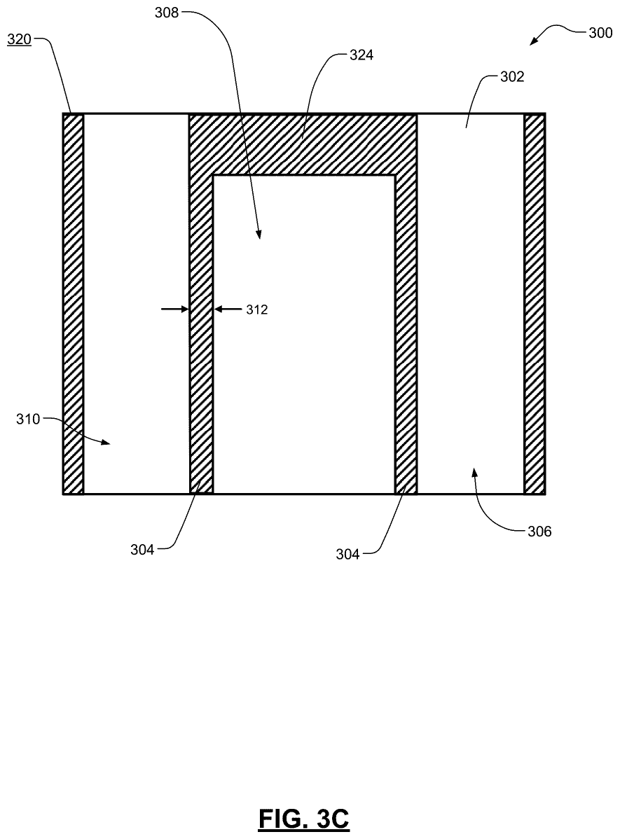

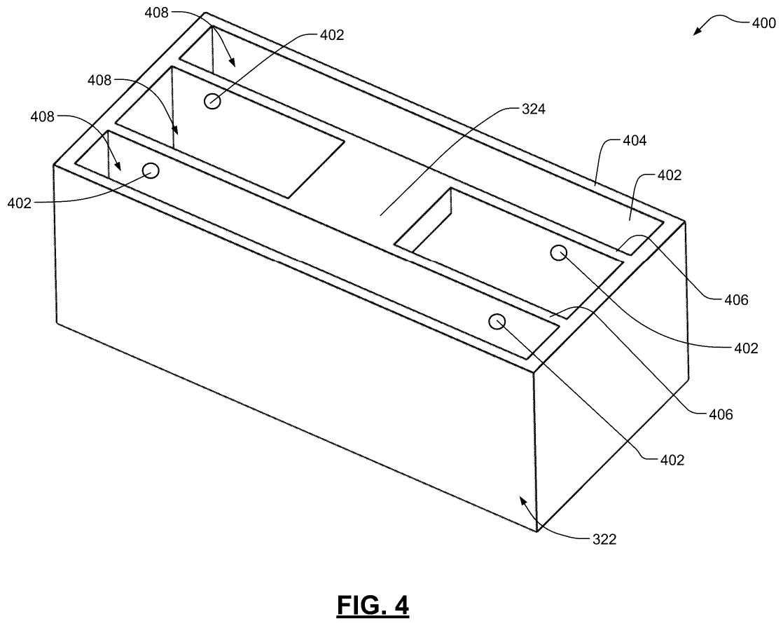

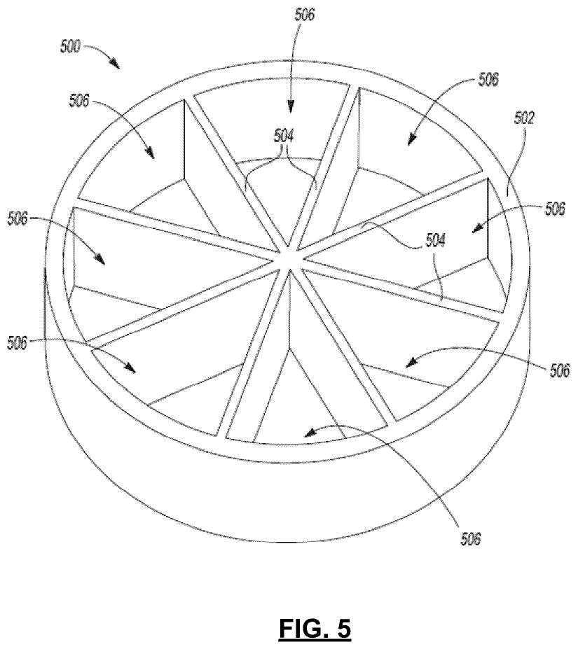

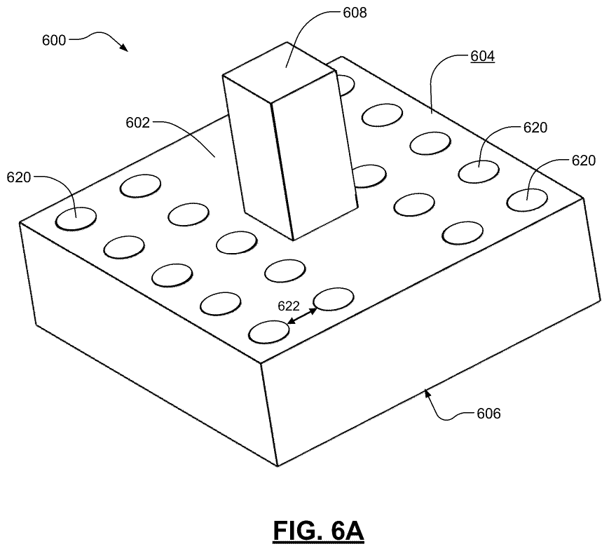

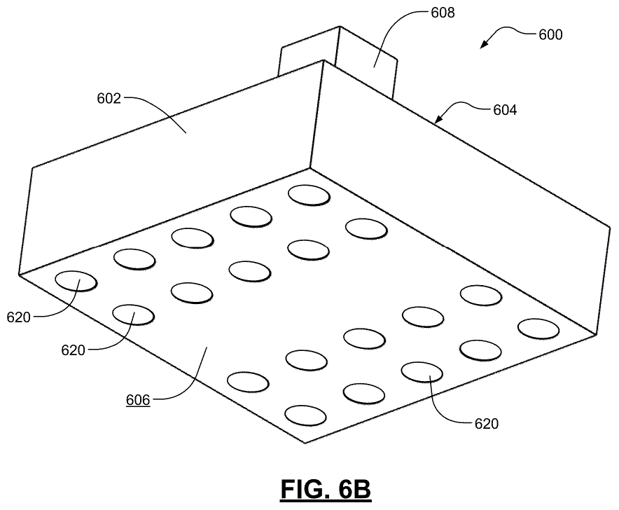

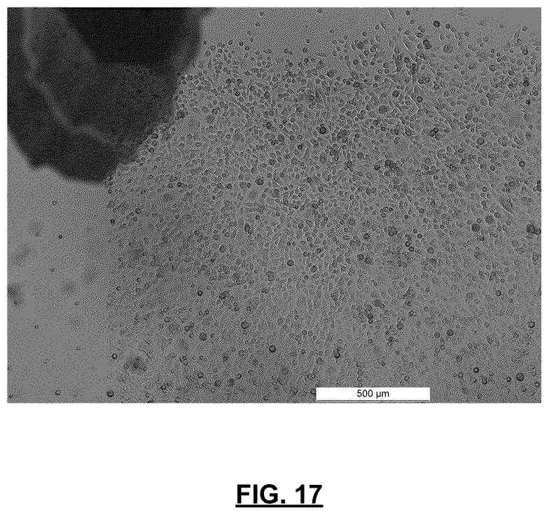

The barrier component 124 may define regions that are polygonal (e.g., rectangular, as in FIG. 1A-1B, 3A-3C, 12A-12C), elliptical (e.g., circular, as in FIG. 6A-6B), wedge-shaped (see, e.g., FIG. 5), irregular (see, e.g., FIG. 16), and/or any other desired shape. Shapes of the regions may be the same as or different from other regions. A Region may be adjacent to another region, nested within another region, protruding into another region. When the barrier component 124 defines multiple regions, each of the regions may be adjacent to one or more of the other regions. The regions may cooperate to form a single layer, 2D culture or a multi-layer 3D culture. 3D cultures may include regions that intersect and/or transect other layers. Additionally or alternatively, a region may form a distinct entity within one or more of the layers.

In at least one example embodiment, the barrier component 124 is a 3D printed barrier component. The barrier component 124 may be formed from a metal (e.g., stainless steel), a polymer (e.g., nylon, high density polyethylene, polypropylene, and/or any other inert material), or a combination thereof. The barrier component 124 may be configured to be sanitized and reused, or alternatively, disposable.

With reference to FIGS. 3A-3E, a barrier component 300 according to at least one example embodiment includes a peripheral wall 302 and transverse walls 304. The peripheral wall 302 and the transverse walls 304 cooperate to at least partially define a plurality of regions. The peripheral wall 302 may surround the plurality of regions.

The plurality of regions may include a first region 306, a second region 308, and a third region 310. The regions 306, 308, 310 may each be substantially rectangular. The first and third regions 306, 310 may have substantially the same size. The second region 308 may be between the first and third regions 306, 310 and have a larger footprint than the first and third regions 306, 310.

In at least the example embodiment shown, the transverse walls 304 extend substantially parallel to one another. The transverse walls 304 may define a thickness 312 (shown in FIG. 3C) of greater than or equal to about 0.1 mm (e.g., greater than or equal to about 0.5 mm, greater than or equal to about 1 mm, greater than or equal to about 2 mm, greater than or equal to about 3 mm, greater than or equal to about 4 mm, greater than or equal to about 5 mm, greater than or equal to about 7 mm, greater than or equal to about 10 mm, greater than or equal to about 15 mm, greater than or equal to about 20 mm, greater than or equal to about 25 mm, greater than or equal to about 30 mm, greater than or equal to about 35 mm, greater than or equal to about 40 mm, greater than or equal to about 45 mm). The thickness 312 may be less than or equal to about 50 mm (e.g., less than or equal to about 45 mm, less than or equal to about 50 mm, less than or equal to about 35 mm, less than or equal to about 30 mm, less than or equal to about mm, less than or equal to about 20 mm, less than or equal to about 15 mm, less than or equal to about 10 mm, less than or equal to about 7 mm, less than or equal to about 5 mm, less than or equal to about 4 mm, less than or equal to about 3 mm, less than or equal to about 2 mm, less than or equal to about 1 mm, or less than or equal to about 0.5 mm). In at least one example embodiment, the regions 306, 308, 310 are separated by a distance that is approximately equal to the thickness.

The barrier component 300 extends between a top surface 320 and a bottom surface 322. The bottom surface 322 is configured to contact and/or engage a plate (e.g., plate 122 of FIGS. 1A-1C), either directly or indirectly (e.g., via a pliable material). The top surface 320 is configured to engage a press (e.g., press 120 of FIGS. 1A-1C), either directly or indirectly (via a barrier cap, as will be described below in relation to FIGS. 9-12C). In at least one example embodiment, the barrier component 300 further includes a contact pad 324 that includes a portion of the top surface 320. The contact pad 324 may facilitate alignment with the press.

Referring to FIG. 4, a barrier component 400 according to at least one example embodiment is provided. The barrier component 400 is the same as the barrier component 300 of FIG. 3 except for the presence of ports 402. The barrier component 400 includes a peripheral wall 404 and transverse walls 406 that cooperate to define a plurality of regions 408. The ports 402 are defined in the transverse walls 408 to fluidly connect the regions 408. In at least one example embodiment, the ports 402 are configured to facilitate osmotic flow of cells between the regions 408 when the regions are filled with a liquid that is in communication with the port.

With reference to FIG. 5, a barrier component 500 according to at least one example embodiment is provided. The barrier component 500 is the same as the barrier component 300 of FIG. 3 except for the shape and arrangement of a peripheral wall 502 and transverse walls 504 that cooperate to define regions 506. Each of the regions 506 defines a wedge shape.

Referring to FIGS. 6A-6B, a barrier component 600 according to at least one example embodiment is provided. The barrier component 600 includes a body 602 that extends between a top surface 604 and a bottom surface 606. In at least one example embodiment, the barrier component 600 further includes a stem 608 extending from the body 602. The stem may facilitate alignment with a press (e.g., press 120 of FIGS. 1A-1C). In at least one example embodiment, the stem is received in a receptacle (e.g., recess) of a barrier cap, as will be discussed in greater detail below in relation to FIGS. 9-12C. In at least one example embodiment, the barrier component 600 may be used for cloning or initial plating of primary cells before transferring them to a second culture dish for continued expansion.

In the example embodiment shown, the body 602 defines a plurality of apertures 620. Each of the apertures 620 may correspond to a region for cell growth. Each of the apertures 620 may extend continuously between the top surface 604 and the bottom surface 606. Each of the regions may be spaced apart from other adjacent regions by a distance 622. Each of the apertures 620 may have substantially the same size and shape as other apertures 620. In at least one other example embodiment, apertures have different sizes and/or shapes. Spacing between the apertures 620 may be uniform or non-uniform.

With reference to FIG. 7, a barrier component 700 according to at least one example embodiment is provided. The barrier component 700 includes a peripheral or outer wall 702 and an inner wall 704. The barrier component 700 further includes a contact pad 706 configured to engage a press (see, e.g., press 120 of FIGS. 1A-1B).

In at least one example embodiment the barrier component 700 defines nested barrier regions. The peripheral wall 702 and the inner wall 704 may cooperate to at least partially define a first region 710. The inner wall 704 may at least partially define a second region 712. The first region 710 at least partially surrounds the second region 712. In the example embodiment shown, the second region 712 is nested within the first region 710.

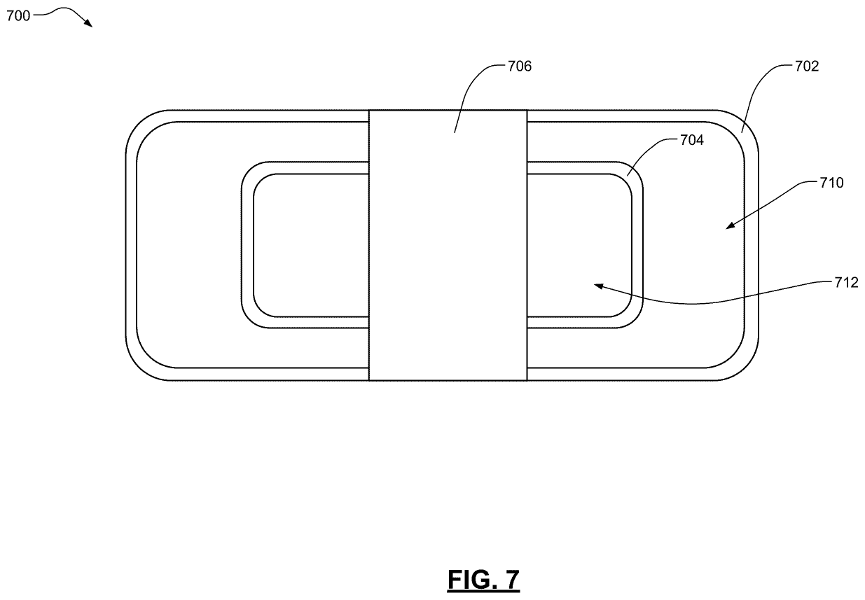

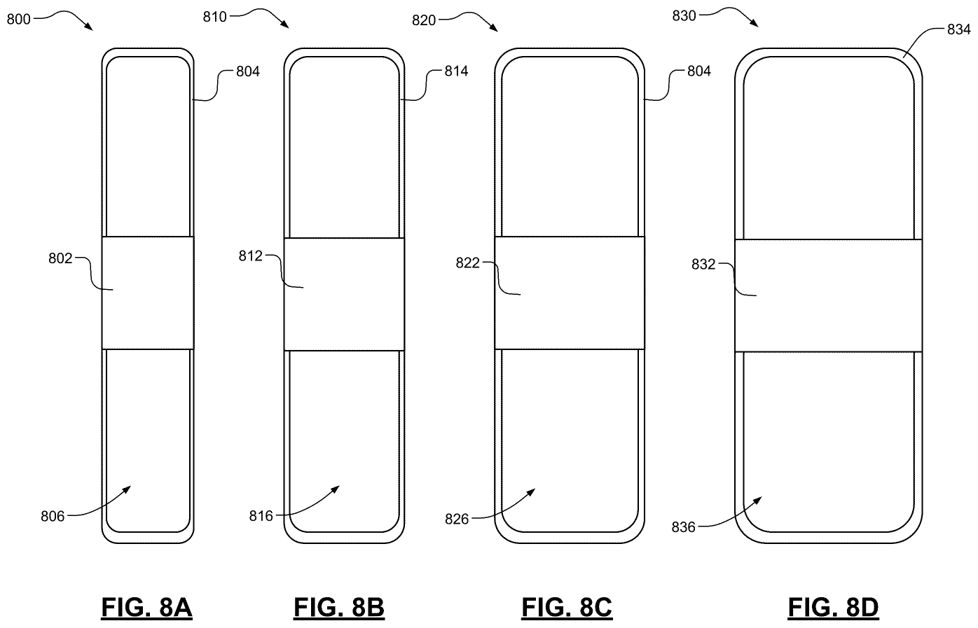

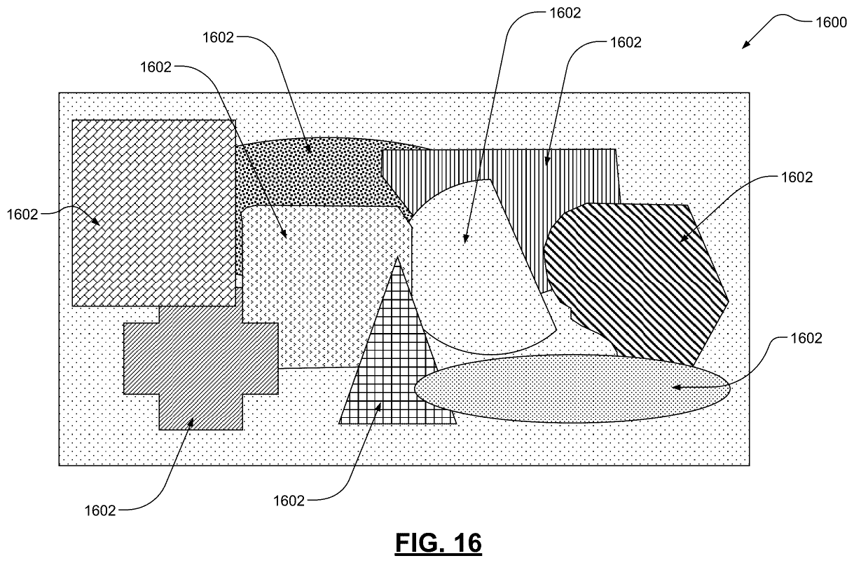

FIGS. 8A-8D provide a plurality of barrier components according to at least one example embodiment is provided. Referring to FIG. 8A, a first barrier component 800 includes a first contact pad 802 and a first peripheral wall 804 that at least partially defines a first region 806. With reference to FIG. 8B, a second barrier component 810 includes a second contact pad 812 and a second peripheral wall 814 that at least partially defines a second region 816. Referring to FIG. 8C, a third barrier component 820 includes a third contact pad 822 and a third peripheral wall 824 that at least partially defines a third region 826. With reference to FIG. 8D, a fourth barrier component 830 includes a fourth contact pad 832 and a fourth peripheral wall 834 that at least partially defines a fourth region 836. In at least one example embodiment, the barrier components 800, 810, 820, 830 may be used to form a multi-laminate, as will be described in greater detail below in relation to FIG. 15.

### Barrier Cap

At least some example embodiments of a barrier apparatus include a barrier cap for facilitating axial alignment of a press and a barrier component and/or a substantially even application of pressure to the barrier component. The barrier cap may be customized together with the barrier component(s) or standard with the apparatus. With reference to FIG. 9, a barrier apparatus 900 according to at least one example embodiment is provided. The barrier apparatus 900 is the same as the barrier apparatus 100 of FIG. 1 except that it is configured for use with a barrier cap, includes a single press, is free of a base recess, and is shown with a different plate and barrier component. The barrier apparatus 900 includes a housing including a base 902 and a cover (not shown). The barrier apparatus 900 further includes a press 904, a culture dish 906, a barrier component 908, and a barrier cap 910. The barrier cap 910 is between the barrier component 908 and the press 904. The barrier cap 910 may be formed from or include a metal (e.g., stainless steel), a polymer (e.g., nylon, high density polyethylene, polypropylene, and/or any other suitable material), or a combination thereof. Materials of a barrier cap and barrier component may be the same or different.

A bottom surface 920 of the barrier component 908 is on a base of the culture dish 906. A top surface 922 of the barrier component 908 engages the barrier cap 910. In at least one example embodiment, a portion of the barrier component 908 including the top surface 922 is in a recess 924 defined by the barrier cap 910. The barrier cap 910 includes a contact pad 926 configured to engage the press 904. The barrier cap 910 facilitates alignment of the press 904, the barrier component 908, and/or the culture dish 906 along a press axis 930. During operation of the apparatus 900, the press 904 is configured to apply pressure to the barrier component 908 along the press axis 930. The press axis 930 extends substantially perpendicular to the top and bottom surfaces 920, 922 of the barrier component 908.

As discussed above, the barrier cap 910 may be configured to facilitate substantially uniform distribution of pressure to the barrier component 908. In at least one example embodiment, the barrier cap 910 is configured to be in contact with greater than or equal to about 30% of an area of the top surface 922 of the barrier component 908 (e.g., greater than or equal to about 40% of the area, greater than or equal to about 50% of the area, greater than or equal to about 60% of the area, greater than or equal to about 70% of the area, greater than or equal to about 80% of the area, greater than or equal to about 90% of the area, or greater than or equal to about 95% of the area). In at least one example embodiment, the barrier cap 910 is configured to be in contact with substantially the entire top surface 922 of the barrier component 908.

In at least one example embodiment, as shown, the barrier cap 910 may define one or more openings 940. The openings 940 be in fluid communication with regions 942 at least partially defined by the barrier component 908 and the culture dish 906. Accordingly, the openings 940 may permit user access to the regions 942, such as for pipetting cell populations into the regions 942.

In at least one example embodiment, the barrier cap 910 includes one or more teeth 950. The base 902 may define a corresponding receptacle 952 configured to receive one of the teeth 950. The receptacle 952 is configured to receive a corresponding tooth 950 to stabilize alignment of the barrier cap 910 and/or reduce or prevent rotation and/or slip of the barrier cap 910. In at least one example embodiment, a base may have multiple receptacles configured to receive respective teeth of a barrier cap (not shown).

Referring to FIG. 10, a barrier apparatus 1000 according to at least one example embodiment is provided. The barrier apparatus 1000 may be the same as the barrier apparatus 900 except that the barrier apparatus 1000 includes a different barrier cap. The barrier apparatus 1000 includes a housing including a base 1002 and a cover (not shown). The barrier apparatus further includes a press 1004, a culture dish 1006, a barrier component 1008 and a barrier cap 1010.

Compared to the barrier cap 910 of FIG. 9, the barrier cap 1010 is configured to be in contact with a smaller portion of a top surface 1020 of the barrier component 1008. This configuration may allow a user to have greater access to regions 1022 at least partially defined by the culture dish 1006 and the barrier component 1008. The barrier cap 1010 may define a plurality of recesses 1024 into which a portion of the barrier component 1008 is configured to be received.

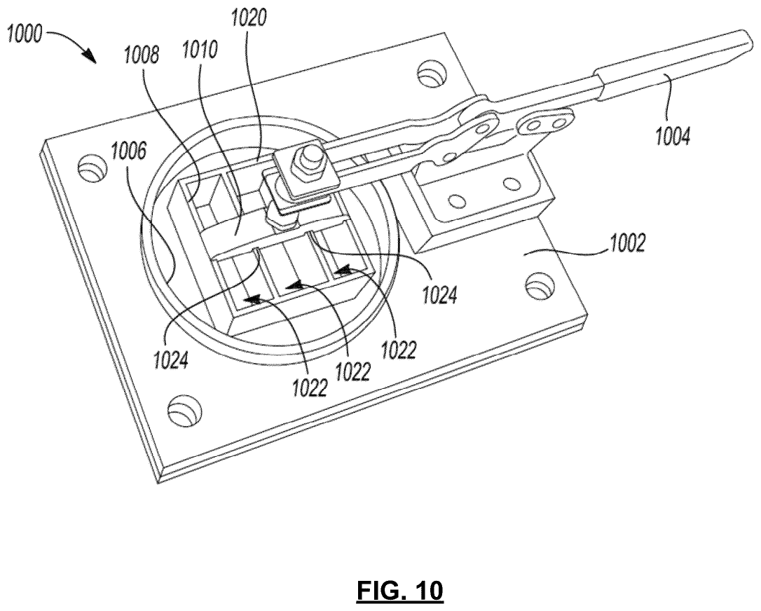

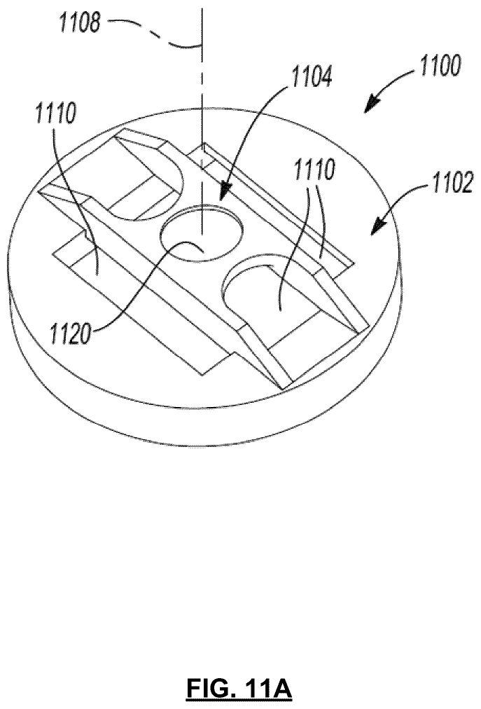

With reference to FIGS. 11A-11B, a barrier cap 1100 according to at least one example embodiment is provided. The barrier cap 1100 includes a main portion 1102 and a bridge portion 1104. The main portion 1102 is configured to receive and/or engage a portion of a barrier component. The main portion 1102 may define a first recess 1106 (shown in FIG. 11B) configured to receive the portion of the barrier component. In at least one example embodiment, the first recess 1106 defines a shape that is substantially the same as a that of the portion of the barrier component to facilitate alignment of the barrier component with the barrier cap 1100 along an axis 1108. In the example embodiment shown, the first recess 1106 defines a substantially rectangular shape. In at least one example embodiment, the main portion 1102 is substantially cylindrical, as shown. The barrier cap 1100 may define a plurality of openings 1110

The bridge portion 1104 is configured to receive and/or engage a portion of a press. In at least one example embodiment, the bridge portion 1104 defines a second recess 1120 (shown in FIG. 11A). The second recess 1120 may be configured to receive a portion of a press to facilitate alignment of the press with the barrier cap 1100 (and therefore also the barrier component) along the axis 1108. In at least one example embodiment, the second recess 1120 defines a substantially circular periphery, as shown. However, a second recess may define any shape suitable for engagement and/or alignment with the press.

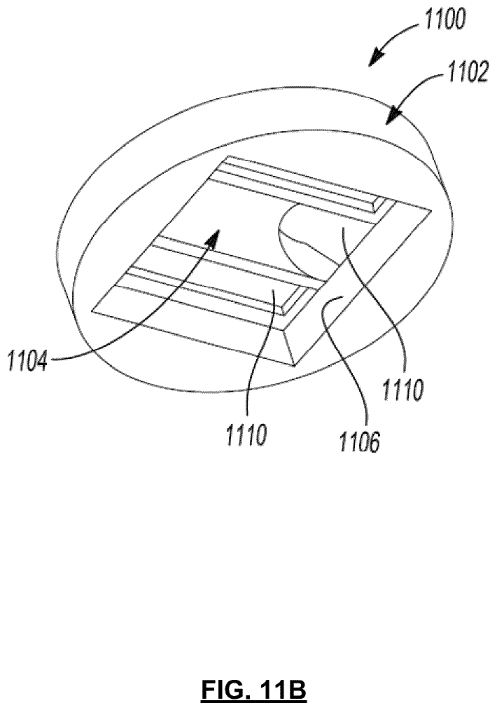

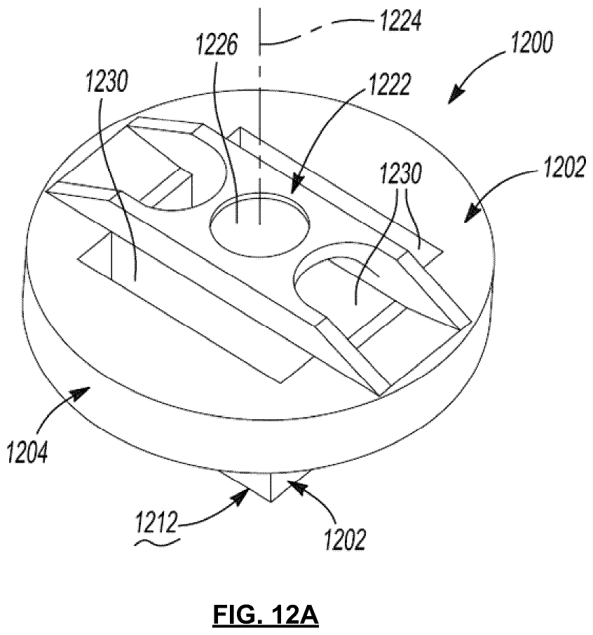

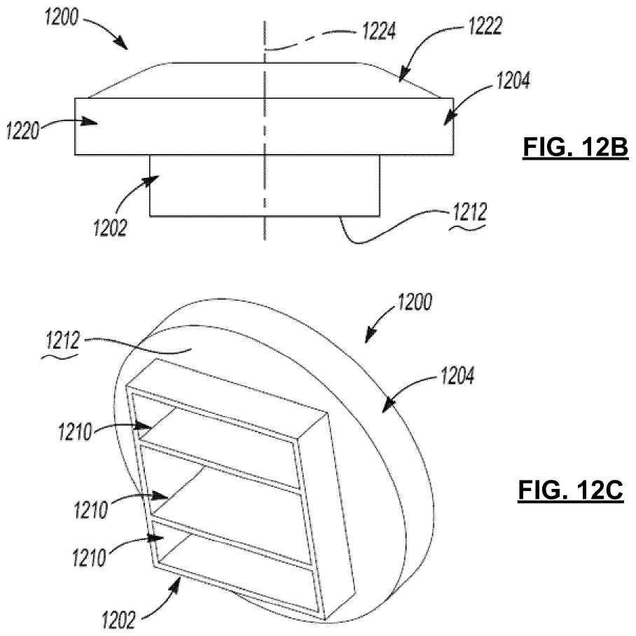

With reference to FIGS. 12A-12C, an integral barrier component-cap 1200 according to at least one example embodiment is provided. The barrier component-cap 1200 may be the same as the barrier cap 1100 of FIG. 11 except that it further includes an integral barrier component. The barrier component-cap 1200 includes a component portion 1202 and a cap portion 1204. In at least one example embodiment, the component and cap portions 1202, 1204 are integrally formed and define a single-piece, unitary structure.

The component portion 1202 at least partially defines a plurality of regions 1210 (shown in FIG. 12C). In the example embodiment shown, the plurality of regions includes three regions. The component portion 1202 includes a bottom surface 1212 configured to contact and/or engage a plate (e.g., a culture dish).

The cap portion 1204 includes a main portion 1220 and a bridge portion 1222. The main portion 1220 is between the bridge portion 1222 and the component portion 1202 along an axis 1224. The bridge portion 1222 defines a recess 1226 configured to receive and/or engage a press (see, e.g., press 904 of FIG. 9). The barrier component-cap 1200 defines a plurality of openings 1230 in fluid communication with the regions 1210.

### Method of Performing Cell Culture

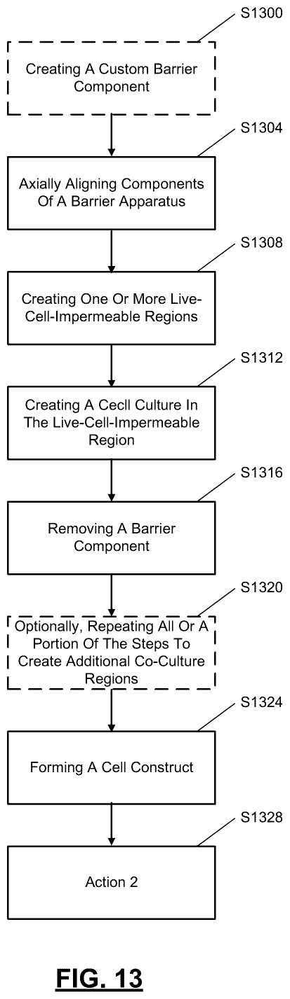

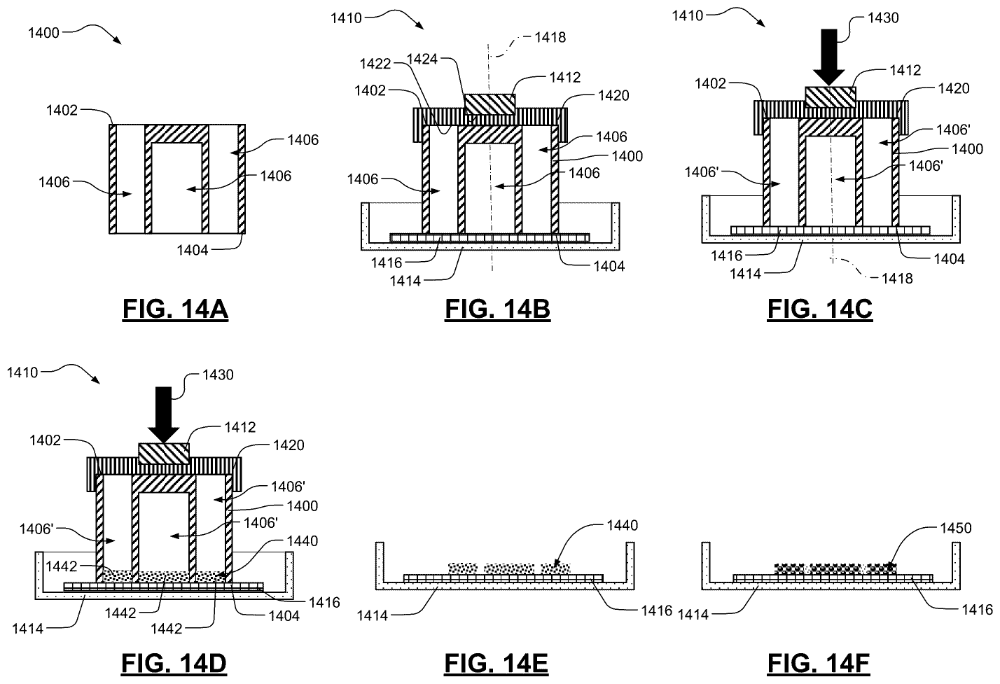

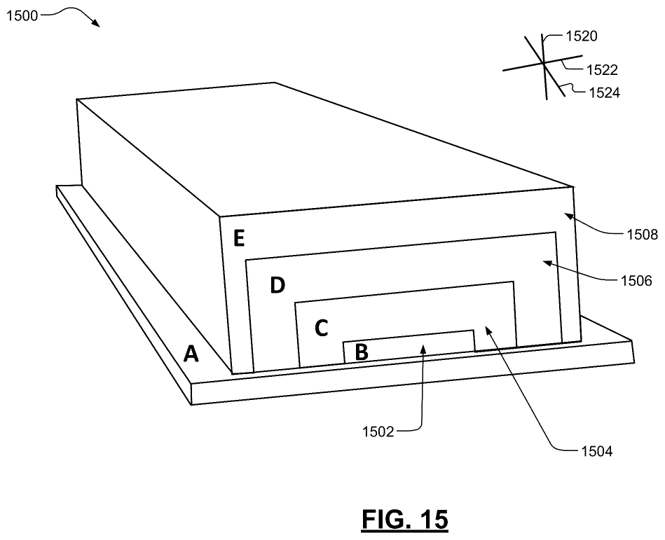

Referring to FIG. 13, a method of performing cell culture according to at least one example embodiment is provided. The method includes optionally creating one or more custom barrier components at S1300; axially aligning components of a barrier apparatus at S1304; creating one or more live-cell-impermeable regions at S1308; creating a cell culture in the region at S1312; retaining the population of cells in the region at S1316; removing a barrier component at S1320; optionally at S1324, repeating all or a portion of steps S1300-S1320; and forming a cell construct at S1328. Each of these steps is described in greater detail below in relation to FIGS. 14A-14F.

### Creating One or More Custom Barrier Components (S1300)

At S1300, the method optionally includes creating one or more custom barrier components. Each barrier component may include as many regions as desired, with each of the regions having a desired shape, placement, volume, footprint, and points of interaction with other regions. The method may include creating a plurality of distinct barrier components for creating a complex multi-laminate, as will be discussed in greater detail in relation to FIG. 15. In one example embodiment, creating the barrier component includes 3D printing. Additionally or alternatively, the barrier component may be manufactured by any other suitable method, such as machining, injection molding, and/or extrusion. The method may optionally further include creating a custom plate and/or barrier cap.

With reference to FIG. 14A, a custom barrier component 1400 according to at least one example embodiment is provided. The barrier component 1400 extends between a top surface 1402 and a bottom surface 1404. The barrier component 1400 at least partially defines one or more regions 1406.

### Axially Aligning Components of a Barrier Apparatus (S1304)

At S1304 (FIG. 13), the method includes axially aligning components of a barrier apparatus. Referring to FIG. 14B, a barrier apparatus 1410 includes a press 1412. A plate 1414 is place in the barrier apparatus 1410. In at least one example embodiment, a pliable matrix or scaffold 1416 (e.g., an acellular dermal matrix, tissue matrix, etc.) may be placed on the plate 1414. The barrier component 1400 is aligned with the press 1412 along a press axis 1418. In at least one example embodiment, a barrier cap 1420 is between the barrier component 1400 and the press 1412 to facilitate axial alignment of the barrier component 1400 and the press 1412. A first recess 1422 of the barrier cap 1420 may receive a portion of the barrier component 1400 and a second recess 1424 of the barrier cap 1420 may receive a portion of the press 1412.

### Creating One or More Live-Cell-Impermeable Regions (S1308)

At S1308 (FIG. 13), the method further includes creating one or more live-cell-impermeable regions S1308. With reference to FIG. 14C, creating live-cell-impermeable regions 1406′ may include applying a predetermined (or alternatively, desired) pressure 1430 to the barrier component 1400 along the press axis 1418 using the press 1412. The barrier apparatus 1410 may be configured to create the live-cell-impermeable regions 1406′ at a joint between the barrier component 1400 and either the pliable matrix 1416 or the plate 1414 directly, such as when the pliable matrix 1416 is absent. The pressure may be selected based on elasticity of a material in direct contact with the bottom surface 1404 of the barrier component 1400 (e.g., the pliable matrix 1416, as shown, or alternatively the plate 1414).

### Creating a Cell Culture in the Live-Cell-Impermeable Region (S1312)

At S1312 (FIG. 13), the method includes creating a cell culture in the live-cell-impermeable region 1406′. Referring to FIG. 14D, creating a cell culture 1440 may include seeding one or more populations of cells 1442 in respective live-cell-impermeable regions 1406′. The populations 1442 may be present in or combined with a liquid culture medium. In at least one example embodiment, the method includes seeding a plurality of populations of cells to create a cell coculture. The method may include maintaining the predetermined pressure 1430 during creating the cell culture.

### Retaining the Population of Cells in the Live-Cell-Impermeable Region (S1316)

At S1316 (FIG. 13), the method includes retaining the population of cells 1442 in the cell-impermeable region 1406′. The cells 1442 may be retained in the respective cell-impermeable regions 1406′ for a first predetermined (or alternatively, desired) period of time. In at least one example embodiment, the first predetermined period of time is selected to allow sufficient time for population growth and/or attachment. The method may include maintaining the predetermined pressure 1430 during S1316.

### Removing the Barrier Component (S1320)

At S1320 (FIG. 13), the method includes removing the barrier component 1400. Removal of the barrier component 1400 may be facilitated by releasing the predetermined pressure 1430, disengaging the press 1412 with the barrier cap 1420 and/or the barrier component 1400, and lifting the barrier component 1400 away from the cell culture 1400. The cell culture 1440 is shown in FIG. 14E.

### Optionally, Repeating Steps (S1324)

At S1324 (FIG. 13), the method optionally includes repeating all or a portion of steps S1300-S1320. For example, such as when the culture has a complex structure including multiple layers that intersect, transect, nest within, and/or otherwise interact with each other, steps S1304-S1320 may be repeated to form additional portions and/or layers of the cell construct. The steps may be repeated using different barrier components.

### Forming a Cell Construct (S1328)

At S1328 (FIG. 13), the method includes forming a cell construct 1450. Forming the cell construct may include retaining the cell culture 1440 on the plate 1414 or another vessel for a second predetermined (or alternatively, desired) period of time to allow the cell culture to mature and/or stratify. The cell construct 1450 is shown at FIG. 14F.

### Example 1

In at least one example embodiment, the method includes creating a mucocutaneous construct (MCC), as described in U.S. patent application Ser. No. 17/540,723 filed on Dec. 2, 2021, which is incorporated by reference herein in its entirety. The method includes providing and/or creating a barrier component. The barrier component defines first, second, and third regions, with the second region being between the first and third regions. The barrier component may be the same as or similar to the barrier component 124 of FIGS. 1A-1C, the barrier component 300 of FIG. 3A-3C, or the barrier component-cap 1200 of FIGS. 12A-12C.

A barrier apparatus according to at least one example embodiment is used to create the MCC. The barrier apparatus generally includes a housing and a press. A culture dish or plate and optionally a barrier cap may be used together with the barrier component in the barrier apparatus. The barrier apparatus may be the same as or similar to the barrier apparatus 100 of FIGS. 1A-1C, the barrier apparatus 900 of FIG. 9, the barrier apparatus 1000 of FIG. 10, and/or the barrier apparatus 1410 of FIG. 14. T

The method may optionally include placing a pliable material, such as an non-immunogenic acellular dermal matrix on the culture dish. The method further includes axially aligning the barrier component with the press such that a bottom of the barrier component engages the culture dish, optionally via the pliable material, and a top of the barrier component engages the press, optionally via the barrier cap. The method further includes applying and retaining pressure to the barrier component via the press (optionally with a barrier cap therebetween) to make the first, second, and third regions substantially impermeable to live cells.

The method further includes creating a coculture while continuing to apply pressure to the barrier component. In at least the example embodiment, creating the coculture includes seeding a first population of skin keratinocytes in the first region and third regions in a first liquid phase. Creating the coculture further includes seeding a second population of mucosa keratinocytes in the second region in the first liquid phase.

The method further includes retaining the coculture in the first liquid phase for a first predetermined period of time while continuing to apply pressure. After the first predetermined period of time, the method includes releasing the pressure and removing the barrier component from the culture dish. The coculture may remain in the culture dish.

The method further includes retaining the coculture in a second liquid phase for a second predetermined period of time. The method further includes forming the MCC by maturing and stratifying the coculture. Maturing and stratifying the coculture may include retaining the coculture in an air-liquid phase for a third predetermined period of time. The MCC includes skin cells in the first and third regions and mucosa cells in the third region.

### Example 2

In at least one example embodiment, a barrier apparatus is used with a plurality of barrier components to create a multi-laminate 1500 as shown in FIG. 15. The multi-laminate 1500 includes a first or innermost portion 1502, a second portion 1504 at least partially surrounding the first portion 1502, a third portion 1506 at least partially surrounding the second portion 1504, and a fourth portion 1508 at least partially surrounding the third portion 1506. Each of the portions 1502, 1504, 1506, 1508 may include different cell types.

The multi-laminate may be formed using a barrier apparatus and the plurality of barrier components of FIGS. 8A-8D. The barrier apparatus may include a barrier cap that is configured to accept each of the barrier components 800, 810, 820, 830 of FIGS. 8A-8D, respectively. For example, the barrier cap may define notches that correspond to each of the respective barrier components.

In at least one example embodiment, a method of making the multi-laminate 1500 includes aligning (e.g., using the barrier cap) and engaging (e.g., applying pressure to) the first barrier component 800 to make the first region 806 impermeable to live cells. The method further includes seeding a first population of cells in the first region and retaining the first population of cells in the first region to allow time for the first population of cells to attach (e.g., overnight or about 12 hours). The method further includes releasing pressure and removing the first barrier component 800.

The method further includes aligning (e.g., using the barrier cap) and engaging the second barrier component 810 to make the second region 816 impermeable to live cells. The first portion 1502 may nest inside of the second region 816. The method further includes seeding a second population of cells in the second region and retaining the second population of cells in the second region to allow time for the second population of cells to attach (e.g., overnight or about 12 hours). The method further includes releasing pressure and removing the second barrier component 810. The second portion 1504 may surround sides and a top of the first portion 1502.

The method further includes aligning (e.g., using the barrier cap) and engaging the third barrier component 820 to make the third region 826 impermeable to live cells. The first and second portions 1502, 1504 may nest inside of the third region 826. The method further includes seeding a third population of cells in the third region and retaining the third population of cells in the third region to allow time for the third population of cells to attach (e.g., overnight or about 12 hours). The method further includes releasing pressure and removing the third barrier component 820. The third portion 1506 may surround sides and a top of the second portion 1504.

The method further includes aligning (e.g., using the barrier cap) and engaging the fourth barrier component 830 to make the fourth region 836 impermeable to live cells. The first, second, and third portions 1502, 1504, 1506 may nest inside of the fourth region 836. The method further includes seeding a fourth population of cells in the fourth region and retaining fourth third population of cells in the fourth region to allow time for the fourth population of cells to attach (e.g., overnight or about 12 hours). The method further includes releasing pressure and removing the fourth barrier component 830. The fourth portion 1508 may surround sides and a top of the third portion 1506. That is, each subsequent portion or layer at least partially surrounds the previous portion or layer in first, second, and third orthogonal directions 1520, 1522, 1524.

### Example 3

With reference to FIG. 16, at least one example embodiment of a multi-laminate 1600 include portions 1602 that differ in terms of shape, volume, and/or footprint. The layers 1602 may be adjacent, nested, intersecting, transecting, or any other desired configuration. The layers 1602 may define footprints having a variety of shapes including, but not limited to, rectangular, triangular, elliptical, circular segments, irregular, cross-shaped, square, or any other desired shape. The multi-laminate 1600 is prepared using a barrier apparatus and one or more barrier components according to at least one embodiment. Laminates according to at least one example embodiment may be used as building blocks with other laminates and/or folded, twisted, rolled on to itself, or otherwise manipulated to connect two or more surfaces to create more complex structures.

### Example 4

With reference to FIG. 17, inverted microscope image of a cell culture after a barrier component is removed according to at least one example embodiment is provided. The left side of FIG. 17 shows an acellular area when compared the center and right side of the image indicating that the barrier is functioning correct. The darker area in the upper left corner shows where the exterior bottom of the culture plate was marked to provide orientation; i.e., the top left corner prior to applying the cells to the interior region created by the barrier.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An apparatus comprising:
a base;
a plate on the base;
a removable barrier component on the plate, the removable barrier component configured to at least partially define a first region, the removable barrier component including,
a bottom surface configured to be in contact with the plate, and
a top surface opposite the bottom surface; and
a press configured to engage the top surface of the removable barrier component to apply a predetermined pressure to the removable barrier component, the removable barrier component being between the press and the plate.

2. The apparatus of claim 1, wherein the removable barrier component is configured to at least partially define a plurality of regions including the first region.

3. The apparatus of claim 2, wherein the removable barrier component further includes a transverse wall between the first region and a second region of the plurality of regions.

4. The apparatus of claim 3, wherein the transverse wall defines a thickness of greater than or equal to about 0.1 mm to less than or equal to about 50 mm.

5. The apparatus of claim 4, wherein the thickness is greater than or equal to about mm to less than or equal to about 5 mm.

6. The apparatus of claim 3, wherein the transverse wall defines a port fluidly connecting the first region and the second region.

7. The apparatus of claim 3, wherein the removable barrier component further includes a peripheral wall surrounding the first region and the second region.

8. The apparatus of claim 2, wherein the first region and the second region are different in terms of projected area, volume, shape, or a combination thereof.

9. The apparatus of claim 2, wherein the second region is nested within the first region.

10. The apparatus of claim 2, wherein the removable barrier component at least partially further defines a third region.

11. The apparatus of claim 1, further comprising:
a plurality of removable barrier components configured to be independently and sequentially disposed on the plate, wherein
the plurality of removable barrier components includes the removable barrier component and a second removable barrier component, and
the second removable barrier component at least partially defines a second region different from the first region.

12. The apparatus of claim 11, wherein the second region at least partially surrounds the first region in first, second, and third orthogonal directions.

13. The apparatus of claim 1, wherein the removable barrier component defines a plurality of wedge-shaped regions including the first region.

14. The apparatus of claim 1, wherein
the removable barrier component defines a plurality of apertures, and
the plurality of apertures includes a first aperture defining the first region.

15. The apparatus of claim 1, further comprising:
a removable cover defining an interior region, the removable cover configured to engage the base to enclose the plate, the removable barrier component, and the press within the interior region.

16. The apparatus of claim 15, wherein the removable cover defines a vent configured to permit air transfer to and from the interior region of the removable cover.

17. The apparatus of claim 15, wherein the base defines a vent configured to permit air transfer to and from the interior region of the removable cover.

18. The apparatus of claim 1, further comprising:
a barrier cap configured to be between the removable barrier component and the press, the barrier cap configured to align the press, the removable barrier component, and the plate along a press axis.

19. The apparatus of claim 18, wherein the barrier cap is configured to engage an entirety of the top surface of the removable barrier component.

20. The apparatus of claim 18, wherein the barrier cap defines an opening in fluid communication with the first region.

21. The apparatus of claim 18, wherein the removable barrier component and the barrier cap are an integrally formed unitary structure.

22. The apparatus of claim 18, wherein the barrier cap defines a recess configured to receive at least a portion of the removable barrier component.

23. The apparatus of claim 1, wherein
the base defines a recess, and
the plate is at least partially in the recess.

24. The apparatus of claim 1, wherein the plate includes glass, plastic, or a combination of glass and plastic.

25. The apparatus of claim 24, wherein a joint between the plate and the removable barrier component is configured to be substantially impermeable to live cells.

26. The apparatus of claim 1, wherein the plate and the removable barrier component are configured to receive a pliable material therebetween.

27. The apparatus of claim 26, wherein a joint between the pliable material and the removable barrier component is configured to be substantially impermeable to live cells.

28. An apparatus for creating cell cultures, the apparatus comprising:
a base configured to support a plate and a removable barrier component that cooperate to define a region for receiving cells, the plate between the base and the removable barrier component;
a press configured to apply pressure to the removable barrier component such that a joint between the plate and the removable barrier component is substantially impermeable to live cells, the removable barrier component being between the press and the plate; and
a removable cover defining an interior region, the removable cover configured to engage the base to enclose the press, the plate, and the removable barrier component within the interior region.

29. A method of creating a cell construct comprising:
applying a first predetermined pressure to a first barrier component using a press, the first barrier component between the press and a plate, the plate between the first barrier component and a base, and the press coupled to the base;
concurrently with the applying, creating a culture by seeding a first population of cells in a first region at least partially defined by the plate and the first barrier component;
concurrently with the applying, retaining the first population of cells in the first region for a first predetermined period of time;
removing the first barrier component from the plate; and
forming the cell construct by maturing the culture for a second predetermined period of time.

30. The method of claim 29, wherein the creating further includes seeding a second population of cells in a second region of the plate, the second region at least partially defined by the plate and the first barrier component, the second region being separated from the first region by a wall of the first barrier component.

31. The method of claim 29, further comprising:
after the removing, placing a second barrier component in contact with the plate, the second barrier component defining a second region at least partially surrounding the first region;
applying a second predetermined pressure to the second barrier component using the press, the second barrier component between the press and the plate;
concurrently with the applying the second predetermined pressure, creating a coculture by seeding a second population of cells in the second region;
concurrently with the applying the second predetermined pressure, retaining the second population of cells in the second region for a third predetermined period of time; and
removing the second barrier component from the plate.

32. The method of claim 29, wherein the culture includes a tissue structure or an organ.

33. A method of creating a mucocutaneous construct (MCC) comprising:
applying a predetermined pressure to a barrier component using a press, the barrier component between the press and a plate, the plate between the barrier component and a base, and the press coupled to the base;
concurrently with the applying, creating a coculture by,
seeding a first population of skin keratinocytes in a first region in a first liquid phase, the first region at least partially defined by the plate and the barrier component, and
seeding a second population of mucosa keratinocytes in a second region in the first liquid phase, the second region at least partially defined by the plate and the barrier component, the second region being separated from the first region by a wall of the barrier component;
concurrently with the applying, retaining the coculture in the first liquid phase for a predetermined period of time;
removing the barrier component from the plate;
after the removing, retaining the coculture in a second liquid phase for a second predetermined period of time; and
forming the MCC by maturing and stratifying the coculture by retaining the coculture in an air-liquid phase for a third predetermined period of time, the MCC including skin cells and mucosa cells.

34. The method of claim 33, further comprising:
prior to the applying, disposing a non-immunogenic acellular dermal matrix on the plate in the first liquid phase such that the non-immunogenic acellular dermal matrix is between the plate and the barrier component.

35. The method of claim 33, wherein a joint between the plate and the wall of the barrier component is configured to be substantially impermeable to live cells during the applying.
